SEQUENCE LISTING (12 sequences)

SEQ ID NO: 1 - DNA, 19 bp, Homo sapiens
ggctgacactctttgtccc

SEQ ID NO: 2 - DNA, 20 bp, Homo sapiens
gtaacatgtacgcctctatc

SEQ ID NO: 3 - DNA, 22 bp, Homo sapiens
gaaccctgctcgaaggctacta

SEQ ID NO: 4 - DNA, 19 bp, Homo sapiens
gctcgaaggctactatgcc

SEQ ID NO: 5 - DNA, 55 bp, Homo sapiens
tggctgacactctttgtcccttcaagagagggacaaagagtgtcagccttttttc

SEQ ID NO: 6 - DNA, 59 bp, Homo sapiens
accgactgtgagaaacagggaagttctctccctgtttctcacagtcggaaaaaagagct

SEQ ID NO: 7 - DNA, 58 bp, Homo sapiens
tgtaacatgtacgcctctatcttcaagagagatagaggcgtacatgttaccttttttc

SEQ ID NO: 8 - DNA, 60 bp, Homo sapiens
acattgtacatgcggagatagaagttctcttatctccgcatgtacaatgaaaaaagagct

SEQ ID NO: 9 - DNA, 62 bp, Homo sapiens
tgaaccctgctcgaaggctactattcaagagatagtagccttcgagcagggttccttttttc

SEQ ID NO: 10 - DNA, 65 bp, Homo sapiens
acttgggacgagcttccgatgataagttctctatcatcggaagctcgtcccaagaaaaaagagct

SEQ ID NO: 11 - DNA, 55 bp, Homo sapiens
tgctcgaaggctactatgccttcaagagaggcatagtagccttcgagcttttttc

SEQ ID NO: 12 - DNA, 59 bp, Homo sapiens
acgagcttccgatgatacggaagttctctccgtatcatcggaagctcgaaaaaagagct

(19) United States
(12) Patent Application Publication
(10) Pub. No.: US 2009/0215863 A1
(43) Pub. Date: Aug. 27, 2009

(54) Gene Silencing of Protease Activated Receptor 1(Par1)

(76) Inventor: Rachel Bar-Shavit, Jerusalem (IL)

Correspondence Address: THE NATH LAW GROUP, 112 South West Street, Alexandria, VA, 22314, US

(21) Appl. No.: 11/990,512

(22) PCT Filed: Aug. 17, 2006
(86) PCT No.: PCT/IL2006/000960

§ 371 Date: May 14, 2008

Related U.S. Application Data
(60) Provisional application No. 60/709,120, filed on Aug. 18, 2005.

(51) Int. Cl. A61K 31/713; C07H 21/02; C12N 15/63; C12N 5/10; A61K 31/711

(52) U.S. Cl. 514/44 A; 536/24.5; 435/320.1; 435/325; 435/366

(57) ABSTRACT

The present invention relates to nucleic acid molecules, vectors, compositions, and methods useful for modulating protease-activated receptor 1 gene expression via RNA interference. In particular, the instant invention features small interfering RNA (siRNA) and short hairpin RNA (shRNA) molecules and methods for modulating the expression of protease-activated receptor 1 gene.

### FIELD OF THE INVENTION

The invention concerns novel nucleic acid molecules for use in gene silencing and RNA interference technologies.

### LIST OF REFERENCES

The following references are considered to be pertinent for the purpose of understanding the background of the present invention:

1. Coughlin S R (2000). Nature 407, 258-4

2. Even-Ram S, Uziely B, Cohen P, Grisaru-Granovsky S, Maoz M, Ginzburg Y, Reich R, Vlodavsky I, Bar-Shavit R. (1998) Nat Med. 4(8):909-14.

3. Even-Ram S C, Grisaru-Granovsky S, Pruss D, Maoz M, Salah Z, Yong-Jun Y, Bar-Shavit R. (2003) J Pathol.; 200(1):47-52.

4. Yin Y J, Salah Z, Grisaru-Granovsky S, Cohen I, Even-Ram S C, Maoz M, Uziely B, Peretz T, Bar-Shavit R. (2003) Arterioscler Thromb Vasc Biol. 23(6):940-4.

5. Martin C B, Mahon G M, Klinger M B, Kay R J, Symons M, Der C J, Whitehead I P. (2001) Oncogene; 20(16): 1953-63

6. Vergnolle N, Cellars L, Mencarelli A, Rizzo G, Swaminathan S, Beck P, Steinhoff M, Andrade-Gordon P, Bunnett N W, Hollenberg M D, Wallace J L, Cirino G, Fiorucci S. (2004) J Clin Invest. 114 (10): 1444-56.

7. Darmoul D, Gratio V, Devaud H, Peiretti F, Laburthe M. (2004) Mol Cancer. Res. 2(9):514-22.

8. Chay C H, Cooper C R, Gendernalik T D, Dhanasekaran S M, Chinnaiyan A M, Rubin M A, Schmaier A H, Pienta K J. Urology (2002) 60(5):760-5.

9. Salah Z, Maoz M, Cohen I, Pizov G, Pode D, Runge M S, Bar-Shavit R. (2005) FASEB J 19(1):62-72.

10. Grisaru-Granovsky S, Salah Z, Maoz M, Pruss D, Beller U, Bar-Shavit R 2005. Differential expression of protease activated receptor 1 (Par1) and pY397FAK in benign and malignant human ovarian tissue samples. Int J Cancer 113(3):372-8.

11. Nierodzik M L, Chen K, Takeshita K, Li J J, Huang Y Q, Feng X S, D'Andrea M R, Andrade-Gordon P, Karpatkin S. (1998) Blood. 92(10):3694-700.

12. Shi X, Gangadharan B, Brass L F, Ruf W, Mueller B M. (2004) Mol Cancer Res. 2(7):395-402

13. Liotta L A, Kohn E C. (2001) Nature 411(6835):375-9.

14. Kalluri R. (2003) Nat Rev Cancer 3(6):422-33.

15. Timpl R. (1996) Curr Opin Cell Biol. 8 (5):618-24

16. Boire A, Covic L, Agarwal A, Jacques S, Sherifi S, Kuliopulos A. (2005) Cell. 120(3):303-13.

17. Zamore P D Nat. Struct. Biol. 2001 8 (9) 746-750.

18. Zhang H., Kolb F A., Brondani V., Billy E., Filipowicz W. EMBO J 21 5875-5885 (2002).

19. Provost P., Dishart D., Doucet J., Frendewey D., Samuelsson B., Radmark O EMBO J (2002)21 5864-5874.

20. Elbashir S M., Harborth J., Lendeckel W., Yalcin A Weber K., Tuschl T Nature (2001) 411 428-429.

21. Miyagishi M, Taira K (2002) Nat. Biotechnol. 20 (5): 497-500.

22. Rubinson D A, Dillon C P, Kwiatkowski A V, Sievers C, Yang L, Kopinja J, Rooney D L, Ihrig M M, McManus M T, Gertler F B, Scott M L, Van Parijs (2003) Nat Genet. 33(3):401-6.

23. Even-Ram S C, Maoz M, Pokroy E, Reich R. Katz B Z, Gutwein P, Altevogt P, Bar-Shavit R. (2001) J Biol Chem.; 276 (14):10952-62

24. Yin Y J, Salah Z, Maoz M, Ram S C, Ochayon S, Neufeld G, Katzav S, Bar-Shavit R. (2003) FASEB J. 17(2):163-74.

25. Hirota Y et al., Journal of Clinical Endocrinology 90(6): 3673-3679, 2005.

26. Parrish et al. 2000, Molecular Cell, 6. 1077-1087.

27. McManus M T, Sharp P A. (2002) Nat Rev Genet 3(10):737-47.

28. Lois C, Refaeli Y, Qin X F, Van Parijs L. (2001) Curr Opin Immunol. 13(4):496-504.

29. Naldini L. (1998) Curr Opin Biotechnol. 9(5):457-63

30. Naldini L, Blomer U, Gallay P, Ory D, Mulligan R, Gage F H, Verma I M, Trono D. (1996) Science.; 272 (5259):263-7.

31. Pfeifer A, Ikawa M, Dayn Y, Verma I M. (2002). Proc Natl Acad Sci USA.; 99(4):2140-5.

32. Lois C, Hong E J, Pease S, Brown E J, Baltimore D (2002) Science. 295(5556):868-72.

33. Salah Z, Maoz M, Cohen I, Pizov G, Pode D, Runge M S, Bar-Shavit R. (2005) FASEB J. 19(1):62-72

34. Fidler I J (1970) J Natl Cancer Inst. 45(4):773-82

35. Esumi N, Fan D, Fidler I J. (1991) Cancer Res.; 51 (17):4549-56.

WO 01/75164

WO 04/005457

WO 06/078798

WO 06/069037

WO 04/028471

WO 97/07387

WO 00/44895

WO 01/36646

WO 99/32619

WO 00/44914

WO 01/68836

WO 04/108897

CA 2,359,180

US 20060142226

US 2006 172963

### BACKGROUND OF INVENTION

Protease Activated Receptors (PARs) are seven transmembrane G-coupled receptors (GPCR) that are uniquely activated by proteolytic cleavage. Four different PARs have been identified (PAR1-4), all responding to a highly select group of serine proteases (1). The PARs act as sensitive sensors of extra cellular protease gradients to allow cells to respond to proteolytically modified environment. While traditionally PAR1 plays a role in thrombosis, hemostasis and vascular biology, it emerges with surprisingly new assignment in tumor biology. This is supported by the pattern of PAR1 expression in normal and pathological epithelia (2-4). In addition, a cDNA expression library screen based on the loss of anchorage-dependent growth and focus forming activity in NIH3T3 cells led to the isolation of PAR1 as a novel oncogene (5). Thus, PAR1 joins a list of GPCRs that are oncogenes including mas and g2α. The transforming activity of PAR1 appears to be due to amplification and ectopic over expression of the gene rather than a constitutively activating mutation. Taken together, the oncogenic properties of PAR1, along with ample evidence on the high expression levels of the human Par1 (hPar1) gene in tumor biopsy specimens and in differentially metastatic cell lines—point to a direct correlation between PAR1 expression and the degree of malignancy. PAR1 has been shown to be involved in a variety of primary human cancers including those of breast (2), colon (6, 7), prostate (8, 9), ovary (10) and melanoma (11, 12).

Malignant tumor growth, metastatic spread and neo vascularization depend on the ability of cells to invade tissue barriers of extra cellular matrix (ECM) and basement membrane structures (13, 14). Basement membranes underlie epithelial and endothelial cell layers and form a structural network composed of typical proteins and polysaccharides (15, 16). Findings by Boire A et al., (16) demonstrate that matrix metalo protease 1 (MMP1) functions as a new class of protease agonist for PAR1. MMP1 activity is mainly derived from stroma fibroblasts. It is secreted to the extra cellular milieu where it is activated.

The fact that PAR1 gene and protein over expression are associated with the aggressiveness of tumors in vivo, reflect on its potential role in tumor dissemination and assigns it as an attractive target for anticancer therapy. In-fact, PAR1 plays a central role at least, in breast tumor progression since introduction of an hPar1 antisense sequence (a plasmid of 462 base pairs antisense sequence containing part of the promoter and the start initiation site of the protein) reduces their ability to migrate through Matrigel coated filters, in vitro (2).

RNA interference (RNAi) refers to the process of sequence-specific post-transcriptional gene silencing mediated by small interfering RNAs (siRNA) (17). The presence of double stranded RNA (dsRNA) in cells triggers various defense mechanisms including the activity of the ribonuclease III enzyme, Dicer. Small RNA duplexes, usually 21 to 23 nucleotides in size, are generated from the dsRNA by the Dicer activity (18, 19). One strand of the duplex (the anti-sense) is then selectively incorporated into a dsRNA-induced silencing complex (RISC) that contains several proteins and guides the selection of a complementary mRNA for cleavage.

Although originally discovered in C. elegans, RNAi has been studied in a variety of systems, including mammalian cells. Elbashir (20) and WO 01/75164 describe RNAi induced by introduction of duplexes of synthetic 21-nucleotide RNAs in cultured mammalian cells including human embryonic cells.

Furthermore, RNAi mediated by siRNA has been proposed as a potential therapeutic modality in a variety of conditions using various genes as the target for silencing; e.g. US 2006 172963 propose RNAi for lowering elevated intraocular pressure to treat patients with glaucoma or ocular hypertension, WO 04005457 use dsRNA to target TCF genes specifically for the treatment of cancer, WO 06078798 use siRNA to modulate the retinoblastoma gene for the treatment of hearing loss, balance disorders and cancer, WO 06069037 use RNAi to inhibit connective tissue growth factor mRNA expression to treat ocular disorders and WO 04028471 provide siRNA compositions for inhibiting influenza.

RNAi may be used to silence gene expression either by administration of the siRNA molecule as such, or by transcription of siRNA from expression vectors and retroviruses capable of infecting mammalian cells.

Retroviruses are easy to manipulate, typically do not induce a strong antiviral immune response, and are able to integrate into the genome of the host cell, leading to stable gene expression. If provided with an appropriate envelope, retroviruses can infect almost any type of cell. Due to these advantages a large number of retroviral vectors have been developed for in vitro gene transfer.

However, vectors based on simple retroviruses are generally unable to integrate into the genome of non-dividing cells, and transgenes expressed from such retroviruses are subject to silencing during development. To overcome these drawbacks, attention has recently focused on lentiviruses which are able to integrate into the genome of non-dividing cells. Accordingly a variety of lentiviral vectors have been developed. For example, Rubinson et al. (22) describe a lentiviral system for delivery of short hairpin RNA (shRNA) into cycling and non-cycling mammalian cells using a U6 promoter (21).

### SUMMARY OF INVENTION

The present invention is based on the finding that gene silencing of protease activated receptor 1 (Par1) expression is useful in manipulation of cancer invasion, metastasis, and angiogenesis wherever PAR1 is involved in these processes. Accordingly, the present invention relates to compounds, compositions and methods useful for modulating Par1 gene expression and/or activity. Specifically, the invention relates to novel small interfering nucleic acid (siNA) molecules, such as small interfering RNA (siRNA) and short hairpin RNA (shRNA) molecules capable of mediating RNA interference (RNAi) against Par1 gene expression. The invention further concerns viral vectors encoding hPAR1 siRNA which enable stable expression and ultimately silencing of PAR1 expression in cancer cells.

Such small nucleic acid molecules and vectors are useful, for example, in providing compositions for treatment of traits, diseases, and conditions that can respond to modulation of PAR1 expression in a subject, such as thrombosis, cardiovascular diseases and cancer e.g. breast, colon, prostate, ovary, and melanoma.

Thus, according to a first aspect, the present invention provides a synthetic siNA molecule, comprising a double stranded portion, having a sequence capable of down regulating expression of human PAR1 (also defined as the thrombin receptor) via RNA interference.

Each strand of the double-stranded portion of the nucleotide molecule comprises, preferably between 18-28 nucleotides. Furthermore, the nucleotide molecule of the invention is preferably a ribonucleic acid (RNA)-based molecule (siRNA).

The siRNA molecule of the invention comprises in one strand of the double stranded portion an antisense region comprising a nucleotide sequence that is complementary to a nucleotide sequence of the hPar 1 gene or a portion thereof, and in the other strand of said double stranded portion a sense region comprising a nucleotide sequence that is substantially similar to a nucleotide sequence of the hPAR 1 gene or a portion thereof. The sense region is complementary to the antisense region.

Specific, albeit non limiting, molecules of the invention comprise, have or are complementary to a sequence selected from the following sequences (all being derived from the RNA sequence of hPar1 (in brackets, the sequence number assigned herein and the corresponding position from hPar1).

(SEQ ID NO: 1, corresponding to a segment starting
from position 542 in hPar1)
5′ GGCTGACACTCTTTGTCCC 3′,
(SEQ ID NO: 2, corresponding to a segment starting
from position 775 in hPar1)
5′ GTAACATGTACGCCTCTATC 3′
(SEQ ID NO: 3, corresponding to a segment starting
from position 1003 in hPar1)
5′ GAACCCTGCTCGAAGGCTACTA 3′
(SEQ ID NO: 4, corresponding to a segment starting
from position 1029 in hPar1)
5′ GCTCGAAGGCTACTATGCC 3′

The nucleic acid molecules of the invention, such as those provided above, may be used as a basis for the formation of siRNA.

According to one embodiment the siRNA of the invention comprises a sequence selected from the sequences depicted in SEQ ID NOs:1 to 4 or the reverse of said sequences.

In one preferred embodiment the sense region of the siRNA molecule is connected to the anti-sense region via a linker, preferably, a polynucleotide linker.

According to this embodiment, the siRNA molecule is assembled from a single nucleotide strand comprising said sense region, said anti-sense region and a polynucleotide linker. Said sense and anti-sense regions of the single strand molecule hybridize to form a double-stranded small hairpin RNA (shRNA) structure.

Specific shRNA of the present invention are provided hereinbelow (marked as SEQ ID NOs:5-12), wherein SEQ ID NOs:1 to 4 (the “sense sequences” or their sequence in reverse) are underlined, the segment consisting of the complementary sequence (the “anti-sense” sequence) is double underlined, and the polynucleotide sequence generating the loop region abridging these two sequences is in italics. The SEQ ID Number of each siRNA is provided in brackets. When the siRNA molecule is assembled from a contiguous single stranded molecule as exemplified below, the “sense” sequences may be provided in reverse depending on their position in the single stranded molecule. Upon formation of the hairpin structure the sense and anti-sense sequences are aligned facing each other and the sense sequence resumes its correct direction.

(SEQ ID NO: 5, sequence underlined once
corresponds to SEQ ID NO: 1)
5′ TGGCTGACACTCTTTGTCCCTTCAAGAGA
GGGACAAAGAGTGTCAGCCTTTTTTC 3′
(SEQ ID NO: 6, sequence underlined once
corresponds to SEQ ID NO: 1 in reverse)
5′ ACCGACTGTGAGAAACAGGGAAGTTCTCT
CCCTGTTTCTCACAGTCGGAAAAAAGAGCT 3′
(SEQ ID NO: 7, sequence underlined once
corresponds to SEQ ID NO: 2)
5′ TGTAACATGTACGCCTCTATCTTCAAGAGA
GATAGAGGCGTACATGTTACCTTTTTTC 3′
(SEQ ID NO: 8, sequence underlined once
corresponds to SEQ ID NO: 1 in reverse)
5′ ACATTGTACATGCGGAGATAGAAGTTCTCT
TATCTCCGCATGTACAATGAAAAAAGAGCT 3′
(SEQ ID NO: 9, sequence underlined once
corresponds to SEQ ID NO: 3)
5′ TGAACCCTGCTCGAAGGCTACTATTCAAGAGA
TAGTALGCCTTCGAGCAGGGTTCCTTTTTTC 3′
(SEQ ID NO: 10, sequence underlined once
corresponds to SEQ ID NO: 3 in reverse)
5′ ACTTGGGACGAGCTTCCGATGATAAGTTCTCT
ATCATCGGAAGCTCGTCCCAAGAAAAAAGAGCT 3′
(SEQ ID NO: 11, sequence underlined once
corresponds to SEQ ID NO: 4)
5′ TGCTCGAAGGCTACTATGCCTTCAAGAGA
GGCATAGTAGCCTTCGAGCTTTTTTC 3′
(SEQ ID NO: 12, sequence underlined once
corresponds to SEQ ID NO: 4 in reverse)
5′ ACGAGCTTCCGATGATACGGAAGTTCTCT
CCGTATCATCGGAAGCTCGAAAAAAGAGCT 3′

The nucleic acid molecules of the invention. such as those provided above may be used for preparing a vector for expressing siRNA effective in down regulating expression of human protein activated receptor (hPAR) in a host cell expressing same.

Thus, by another aspect, the present invention provides novel lentiviral vectors expressing siRNA specific for hPar1.

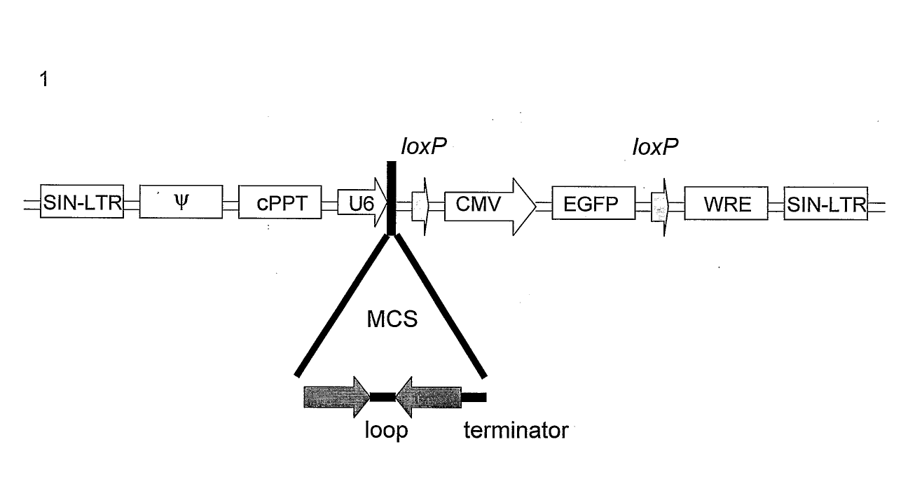

According to one preferred embodiment of the invention, the expression vector is the lentivirus-based vector pLL3.7 (FIG. 1) expressing siRNA, specific for hPar1 under the control of U6 promoter.

According to the invention, transcription directed by the promoter results in synthesis of one or more RNAs comprising complementary (sense and antisense) regions that self-hybridize or hybridize to each other to form a short hairpin RNA (shRNA) or short interfering RNA (siRNA) that inhibit expression of hPar1. When a single RNA molecule is transcribed from the vector and comprises complementary regions that hybridize with each other, the RNA will be said to self-hybridize.

In certain embodiments of the invention the lentiviral vector comprises a nucleic acid segment flanked by two promoters in opposite orientation, so that transcription from the promoters results in synthesis of two complementary RNAs that hybridize with each other to form a siRNA targeted to hPar1. In certain embodiments of the invention the lentiviral vector comprises at least two promoters and at least two nucleic acid segments, so that transcription from the promoters results in synthesis of two complementary RNAs that hybridize with each other to form a siRNA targeted to hPar1. According to certain embodiments of the invention the siRNA or shRNA comprises a double stranded (base-paired) region between 18-28 nucleotides long, preferably approximately 19 nucleotides long.

According to a preferred embodiment the present invention provides pLL3.7 vectors expressing any of the sequences denoted as SEQ ID NOs: 5-12. These sequences comprise sequences of about 19 nucleotides (SEQ ID NO:1-4) of the hPar1 coding region. The resultant expressed siRNAs are in the form of a stem-and-loop (short hairpin) double stranded oligonucleotides.

The invention also provides a host cell transfected with an expression vector encoding the siRNA.

The invention also provides a pharmaceutical composition comprising as active ingredient one or more nucleotide molecules, or siRNA of the invention, or the expression vector encoding the latter, the nucleotide molecules, siRNA or expression vector being in a form suitable for cell transfection.

As mentioned above, the siRNA of the invention is effective in down regulating expression of Par. gene, thereby inhibiting the production of PAR. Thus, the siRNA of the invention may have a therapeutic benefit in preventing or treating diseases associates with expression of Paw genes, particularly, with over-expression of Par genes. Thus, the pharmaceutical composition of the invention may be useful for the treatment or prevention of a disease or disorder associated with. over-expression of the Par gene, preferably, the Par1 gene.

PAR1 has been shown to be involved in tumor progression by promoting cell invasion/migration and angiogenesis (23, 24). Its role in inflammation, inducing a variety of pro-inflammatory factors such as IL-8, COX-2 MCP1 and TF has also been widely described (25, 6). WO97/07387 whose content is incorporated herein by reference discloses that a direct correlation exists between PAR1 (thrombin) level of expression in tumor cells and their degree of invasiveness. Thus, without being limited thereto, the siRNA of the invention may be useful in the treatment or prevention of cancer, including growth, dissemination, and metastasis thereof, angiogenesis, inflammation, and hemostasis.

A specific application of the siRNA of the invention is in the prevention of cancer dissemination and metastasis of invasive cancer cells. In one embodiment, the invasive cells are metastatic tumor cells of epithelial origin, which form solid carcinoma-type tumors (e.g. melanoma). Examples of such epithelial tissues are breast, esophagus, kidney, prostate, ovary, and bladder tissue.

In one embodiment of the invention the expression vector comprises a promoter that is selectively activated in specific cells, thus endowing the vector with target specificity. Examples of such cell specific promoters are abundant in the art. One such non-limiting example is the MMTV promoter that is used to direct heterologous vector expression in breast cancer cells.

The invention also provides a method for the treatment or prevention of a disease or disorder associated with over-expression of the PAR1 gene in a subject, the method comprises providing said subject with an amount of a nucleic acid molecule comprising the siRNA of the invention, or the expression vector, the amount being sufficient to inhibit expression of said gene.

Finally, the invention provides the use of the above defined nucleotide molecule, the siRNA or vector expressing the latter, for the manufacture of pharmaceutical compositions, preferably for treating conditions associated with the Par1 gene.

### DETAILED DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows the general constituents of U6 promoter-driven lentivirus (pLentilox 3.7) vector of hPar1 siRNA according to one embodiment of the invention. SIN-LTR, self-inactivating long terminal repeat; Ψ, HIV packaging signal; cPPT, central polypurine track; U6, U6 promoter; MCS, multiple cloning site; CMV, cytomegalovirus promoter; EGFP, enhanced fluorescence protein; WRE, woodchuck hepatitis virus response element.

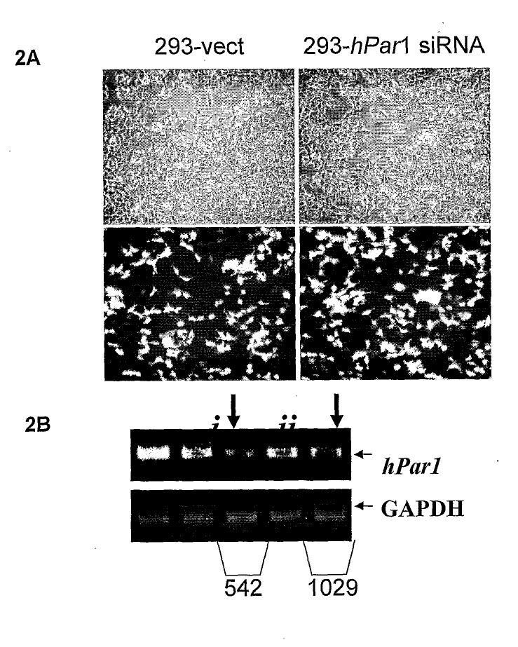

FIGS. 2A-2B show levels of Par1 mRNA in cells transfected with pSPi1 or pSPi2 vs. empty vector. FIG. 2A: 293T cells transfected either with GFP-empty vector (293-vec) or hPar1 siRNA-GFP lenti-vector (293-hPar1 siRNA). Top: Phase contrast microscopy Bottom: Fluorescent microscopy showing the yield of infection. FIG. 2B: Semi-quantitative RT-PCR showing levels of hPar1 in the absence or in the presence of 4 siRNA hPar1 U6 promoter-driven lentivirus (pLentilox 3.7) vector (pLi, pL542, pLii, pL1029) the GAPDH gene served as the control.

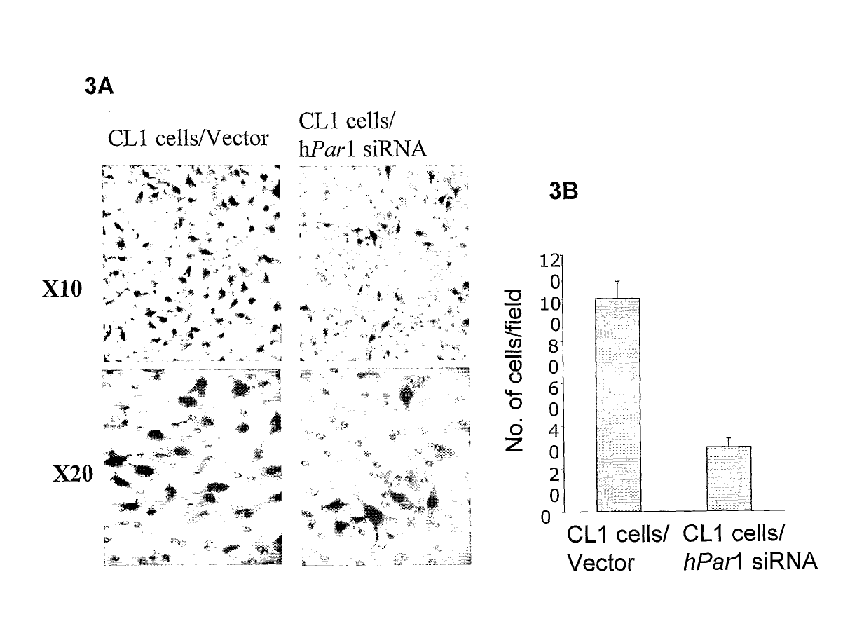

FIGS. 3A-3B show inhibition by hPar1 siRNA of CL1 cells invasion through Matrigel-coated filters vs. empty vector. FIG. 3A—Abundant levels of cells invaded the Matrigel coated filters in CL1 highly malignant prostate cells (CL1 cells/vector) showing a markedly inhibited invasion in the presence of hPar1 siRNA (CL1 cells/hPar1 siRNA)×10 magnification. Bottom panel shows the same as the top panel but at ×20 magnification. FIG. 3B—is a histogram illustrating the number of invading cells under these (as in A) conditions.

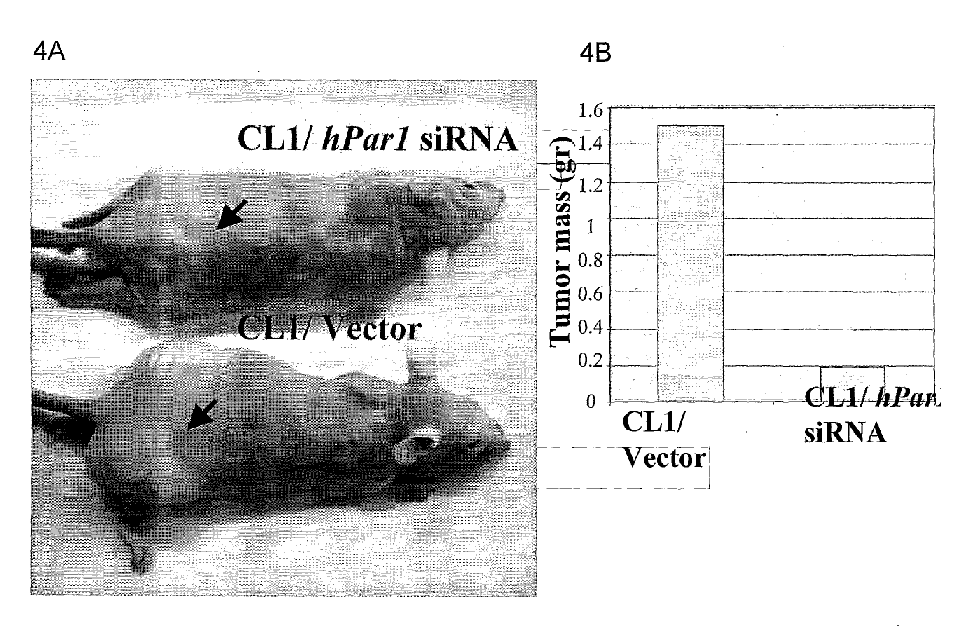

FIGS. 4A-4B show hPar1 siRNA effect on tumor growth, FIG. 4I is a representative photograph of mice injected subcutaneously (sc) with CL1 cells that have been infected with either hPar1 siRNA vector (upper) or with control vector (lower); FIG. 4II tumor mass of CL1 cells infected with hPar1 siRNA vector relative to the control vector.

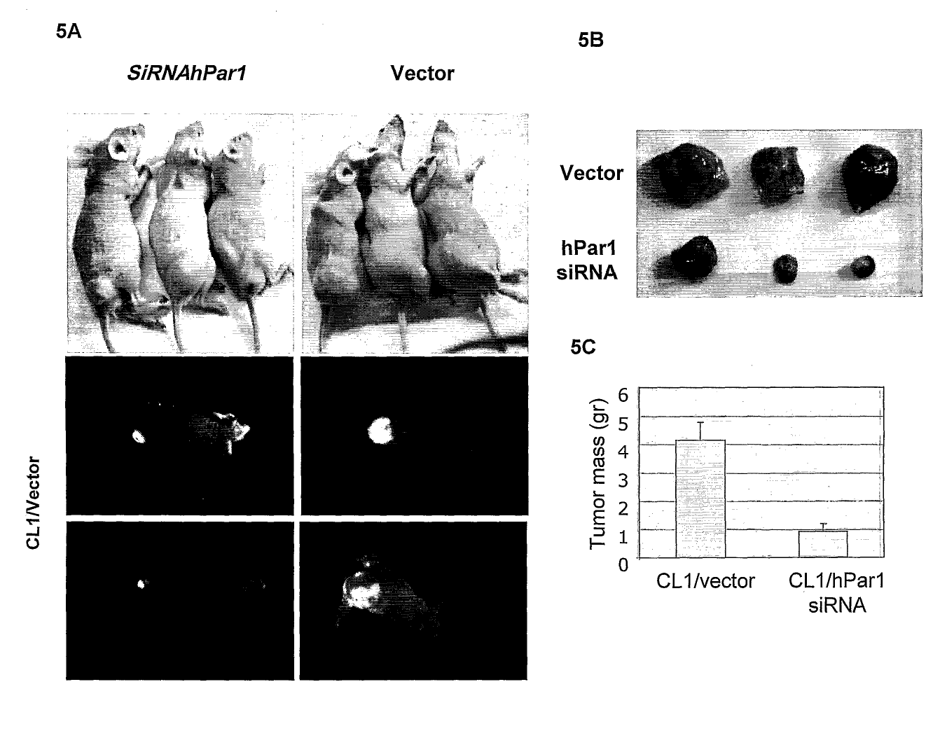

FIGS. 5A-5C show the results of GFP-hPar1 siRNA infected CL1 cells injected subcutaneously (sc) to SCID mice vs. injection of empty vector. FIG. 5A—Tumors formed in CL1 injected mice (vector) and in hPar1 siRNA infected CL1 cells (hPar1 siRNA)—top panel. Fluorescence imaging of GFP-positive tumors in intact mice—Lower panel. Right—CL1 cells, Left—hPar1 siRNA mice. FIG. 5B—Tumors excised from either vector infected CL1 cells (top) or hPar1 siRNA (bottom). FIG. 5C—Histogram illustrating the weight of tumors formed by infection of CL1/vector or CL1/hPar1 siRNA.

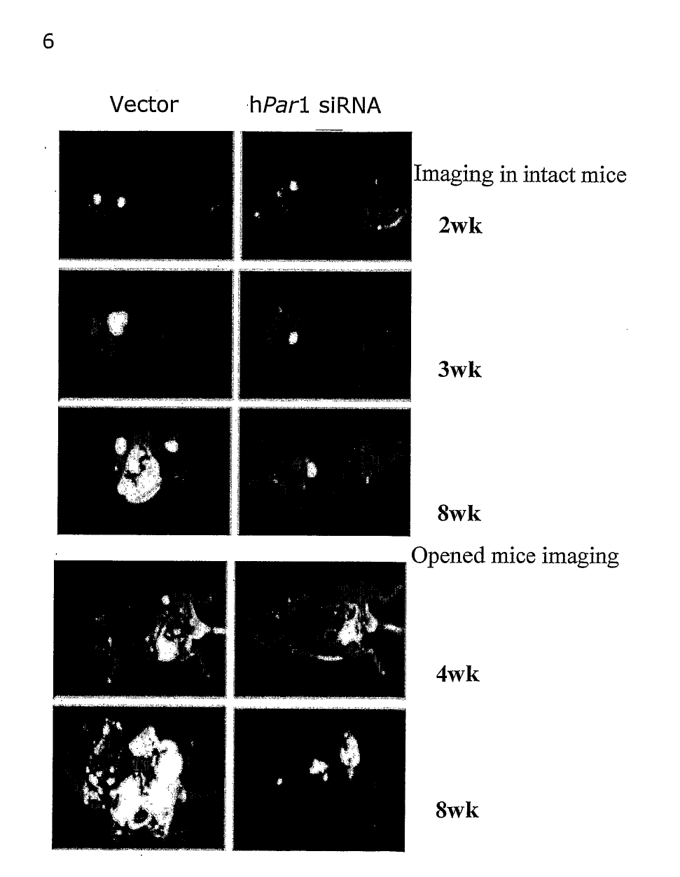

FIG. 6—shows representing photographs of mice injected orthotopically into the prostate gland lobes with CL1 cells that have been infected with either hPar1 siRNA viral vectors (right) or with an empty control vector (left). Mice were analyzed at various time points as indicated on the left.

### DETAILED DESCRIPTION OF EXEMPLARY EMBODIMENTS OF THE INVENTION

Some terms used herein and their meanings are as follows:

Down regulating—Reducing the expression of a gene, or levels of RNAs or activity of a protein such as hPAR-1 below that observed in the absence of the nucleic acid molecules of the invention.

Modulation—Up regulation or down regulation of the expression of the gene, or level of RNAs or activity of a protein such as hPAR-1, such that the expression, level or activity is greater than or less than that observed in the absence of the nucleic acid molecules of the invention.

Nucleic acid molecule—A molecule having nucleotides, being either single or double stranded, and can comprise modified or unmodified nucleotides or non-nucleotides or various mixtures thereof.

Ribonucleic acid (RNA)-based molecule—A molecule comprising at least one ribonucleic acid residue, but may comprise a combination of ribonucleic and deoxyribonucleic acids (DNA) as well as chemically modified and/or synthetic nucleotides as known to those versed in biotechnology. It is preferable however, that at least the majority of the nucleotides in the molecule are RNAs or RNA derivatives. The term “majority” in accordance with the invention denotes at least 85%, preferably 95% and most preferably between 99%-100%. Replacing up to four nucleotides on each end of the siRNA with deoxyribonucleic acids has been reported to be well tolerated, whereas complete substitution with deoxyribonucleic acids results in no RNAi activity (20; WO 01/75164).

Double stranded RNA or dsRNA—a double stranded RNA that matches a predetermined gene sequence that is capable of activating cellular enzymes that degrade the corresponding messenger RNA transcripts of the gene. These dsRNAs are referred to as short interfering RNA (siRNA) and can be used to inhibit gene expression via RNA interference (RNAi). (For example: Elbashir et al. (20); WO 00/44895; WO 01/36646; WO 99/32619; US 20060142226). An siRNA may be formed from two RNA strands that hybridize together, or may alternatively be generated from a single RNA strand that includes a self hybridizing portion.

RNA interference—the process of sequence-specific post-transcriptional gene silencing mediated by small interfering NAs (siNA), specifically siRNA.

Short interference—an RNA molecule comprising complementary sense and antisense portions hybridized or capable of hybridizing to form a double-stranded structure sufficiently long to mediate RNAi (typically approximately 19 base pairs in length), and at least one single-stranded portion, typically between approximately 1 and 10 nucleotides in length that forms a loop. The loop portion of the hairpin siRNA molecule can comprise nucleotides, non-nucleotides, linker molecules, or conjugate molecules. shRNAs are thought to be processed into siRNAs by the conserved cellular RNAi machinery. Thus shRNAs are precursors of siRNAS and are similarly capable of inhibiting expression of a target manuscript, e.g. hPar1.

Duplex—a siNA molecule having two separate strands comprising a sense region and an antisense region, wherein the sense region has enough complementary nucleotides to base pair with the antisense region and form a duplex. For example, a duplex siNA molecule of the invention can comprise an antisense region having length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a sense region having about 15 to about 30 (or about 15, 16, 17, 18, 19, 20, 21, 22, 23, 24, 25, 26, 27, 28, 29, or 30) nucleotides that are complementary to the antisense region.

Complementary nucleic acid—A nucleic acid capable of forming hydrogen bonds with another nucleic acid sequence by either traditional Watson-Crick or other non-traditional types of interaction. The degree to which one nucleic acid is complementary to another is reflected in the percentage of contiguous residues in a nucleic acid molecule capable of forming hydrogen bonds (e.g. Watson-Crick base pairing) with the second nucleic acid sequence (e.g. 5, 6, 7, 8, 9, 10 out of 10 being 50%, 60%, 70%, 80% 90% and 100% complementary).

Sense region—a nucleotide sequence of a siNA molecule complementary to an antisense region of the siNA molecule. In addition, the sense region of a siNA molecule can comprise a nucleic acid sequence having homology with a target nucleic acid sequence.

Antisense region—a nucleotide sequence of a siNA molecule complementary to a target nucleic acid sequence. In addition, the antisense region of a siNA molecule can optionally comprise a nucleic acid sequence complementary to a sense region of the siNA molecule.

Delayed sequence—a sequence corresponding (i.e. identical) to a continuous segment of hPAR1, a complementary of such a segment, or an analog of such a segment. The analog, in accordance with the invention, denotes a molecule comprising a sequence corresponding to a segment of hPAR1 with the exception that one or more nucleotides have been added, deleted or replaced with another nucleic acid (conservative as well as non-conservative replacement). In any case, it is to be understood that the analog is at least 80% identical to the hPar1 segment to which it corresponds, preferably 90% identical, more preferably 95% identical and most preferably having 99-100% identity therewith.

Hybridize—the interaction between two complementary nucleic acid sequences. Guidance for performing hybridization reactions is abundant in the art.

Vector—a nucleic acid molecule capable of mediating entry (e.g. transferring, transporting) of another nucleic acid molecule into a cell. The transferred nucleic acid is generally linked to (e.g. inserted into) the vector nucleic acid molecule. A vector may include sequences that direct autonomous replication, or may include sequences sufficient to allow integration into host cell DNA. Useful vectors include, for example, plasmids, cosmids, and viral vectors. Useful viral vectors include, e.g. replication defective retroviruses, adenoviruses, adno-associated viruses, and lentiviruses.

The present invention features nucleic acid-based molecules, in particular double-stranded small interfering siRNA molecules and methods to modulate the expression of genes encoding for a member of the human protease activated receptor (PAR) family, specifically PAR1, via RNA interference (RNAi). RNAi has emerged as a rapid and efficient way to modulate gene functions in mammalian cells. An advantage of siRNA approach is that one siRNA molecule can cleave many copies of a target mRNA. Thus, relatively low levels of siRNA may achieve efficient inactivation of a targeted gene.

According to a preferred embodiment, the nucleotide molecule of the invention is derived from the hPar1 RNA sequence.

Specific non-limiting examples of the siRNA molecules of the invention comprise siRNA specific for human PAR1, e.g. nucleic acids comprising sequence ID numbers 1-12.

The siRNA molecules of the invention can be un-modified or chemically modified using methods well known in the art without compromising their RNAi activity (e.g. WO 00/44914; WO 01/68836; CA 2,359,180; Parrish et al. (26)). Such modifications may improve various properties of the native siRNA molecules and are of special significance when the molecules are used in vivo, as such. Such modifications may cause an increased resistance to nuclease degradation, or increased cellular uptake.

One potential method of introducing synthetic siRNA into cells is by lipofection. However this method results in low transfection efficiencies and/or short term persistence of silencing effects.

According to one embodiment of the invention, an expression vector is used to express the siRNA molecules in a target cell. Many prokaryotic and eukaryotic expression vectors are known and/or commercially available. Selection of appropriate expression vectors is within the knowledge of those having skill in the art.

An example of a suitable vector and a method for its preparation are provided in WO04108897. The disclosed method includes (a) providing a DNA sequence comprising a segment encoding a strand of the siRNA and a loop segment at the 3′ end, (b) extending the 3′ end of the loop with a polymerase to form a second DNA molecule duplexed with the first DNA molecule, the second DNA molecule encoding the complementary strand of the siRNA, (c) denaturing the duplexed first and second DNA molecules to form a single-stranded DNA molecule comprising the second DNA molecule fused to the 3′ end of the first DNA molecule; (d) synthesizing a second strand complementary to the single-stranded DNA molecule fanning a double stranded DNA molecule; and (e) cloning at least a fragment of the double stranded molecule into a vector in operable linkage with a promoter whereby transcription of the vector forms a transcript comprising a segment having the siRNA strand sequence, the loop segment or its complement, and the complement of the siRNA strand, and the transcript self-anneals to form the siRNA.

Other examples of suitable expression vectors are retroviral vectors which are potent, stable gene delivery tools for use in transfection of mammalian cells (22, 27, 28). The vector chosen for use herein was a lentivirus-based vector (pLL3.7, FIG. 1) that expresses RNAi-inducing short hairpin RNA (shRNAs) for human and mouse Par1 under the control of U6 promoter. This vector engineered to co-express enhanced fluorescent protein (EGFP) as a reporter to permit infected cells to be tracked by flow cytometry. Lentilvirus vectors have two key advantages over other gene delivery system. First, they can infect non-cycling and post mitotic cells (29, 30). Second, transgenes expressed from lentilviruses are not silenced during development and can be used to generate transgenic animals through infection of embryonic stem (ES) cells or embryos (31, 32).

Data from Rubinson et al. (22) demonstrated that lentilvirus can be used to deliver sh RNAs and reduce gene expression in cycling and non-cycling cells, as well as chimeric and transgenic mice. This technology should allow systematic genetic analysis in most cell types and tissues, including those of human origin, and facilitate comprehensive studies of gene function in mice and in species that are not traditionally amenable to genetic manipulation. Lentilvirus expression vectors might be used therapeutically to silence disease-causing genes. Future modifications to lentilvirus expression vectors, such as the inclusion of inducible or tissue specific promoters will extend the range of cells and situations in which they can induce RNAi.

U6 promoter-driven lentivirus (pLentilox 3.7) vector of SiRNA, specific for hPar1 is one preferred embodiment of the invention. According to this specific embodiment sequences of 19 nucleotides (SEQ ID NO:1-4) of the hPar1 coding region were selected for preparation of stem-and-loop siRNA (SEQ ID NO: 5-12).

It may be desirable to achieve cell type specific or tissue-specific expression of hPar1. This can be achieved by insetting into the vector of the invention tissue-specific promoters. As used herein, the term “tissue specific promoter” refers to a regulatory element (e.g., promoter, promoter/enhancer or portion thereof) that preferentially directs transcription in only a subset of cell or tissue type. A tissue specific promoter may direct transcription in only a single cell type or in multiple cell types. Numerous tissue-specific promoters are known, and one of ordinary skill in the art will readily be able to identify tissue specific promoters from the literature. For example, the nestin, neural specific enolase, NeuN, and GFAP promoters direct transcription in various neural or glial lineage cells; the keratin 5 promoter directs transcription in keratinocytes; the MyoD promoter directs transcription in skeletal muscle cells; the insulin promoter directs transcription in pancreatic beta cells; the CYP450 3A4 promoter directs transcription in hepatocytes. The invention therefore provides hPar1 expression vectors as described above further comprising a tissue-specific promoter and methods of using the hPar1 expression vectors and shRNA derived thereof to achieve cell type or tissue specific inhibition of hPar1 expression via RNAi.

Highly metastatic cancer cell lines were transfected with the hPAR1 siRNA construct and examined in vitro, in Matrigel invasion assay, and in vivo, in experimental animal models, whereby the highly aggressive tumor cells were injected into animals in the presence or absence of hPAR1 siRNA and their respective size and metastatic profile (metastasis in the lung, liver and bones) were quantified. hPAR1 siRNA expression markedly attenuated tumor growth. hPar1 siRNA also was shown to significantly reduce tumor angiogenic blood vessel formation of both sprouting and mature blood vessels.

### EXAMPLE 1

### Generation of a U6 Promoter-Driven Lentivirus (pLentilox 3.7) Vector Expressing SiRNA Specific for hPar1

Sequences of 19 nucleotides of the hPar1 coding region were selected for stem-and-loop oligo nucleotide siRNA formation. The selected sequences were submitted to aBLAST search against the human genome sequence to ensure that additional human genes are not targeted. The selected sequences are presented in Table 1:

TABLE 1
hPar1 SiRNA Sequences

| | | |
|---|---|---|
| START | SEQUENCE | SEQ ID NO: |

| | | |
|---|---|---|
| 542 | 5′ ggctgacactctttgtccc 3′ | 1 |
| 775 | 5′ gtaacatgtacgcctctatc 3′ | 2 |
| 1003 | 5′ gaaccctgctcgaaggctacta 3′ | 3 |
| 1029 | 5′ gctcgaaggctactatgcc 3′ | 4 |

To construct the hairpin, stem-and-loop SiRNA expression cassette appropriate DNA nucleotide molecules were synthesized. The nucleotide molecules were composed of the following: 19 nucleic acid bases of the hPar1 coding sequence (sense strand), a loop sequence linker (comprising 9 bases), a reverse complement of the 19 bases of hPar1 coding region (anti-sense strand), and a terminator sequence of poly T. Sticky end of Xho I site was added to the anti-sense strand. Both sense and anti-sense sequences were phosphorylated at the 5′ ends. The sense sequence nucleotides were then annealed to their respective, complementary anti-sense nucleotides. The siRNA cassette sequences were then ligated into the pLentilox3.7 vector (Van Parijs Laboratory). Four such SiRNA cassettes were created from the hPar1 gene targeted toward the Par1 gene sequences shown in Table 1 above.

The resulting lentiviral vectors were analyzed for successful cloning of the siRNA inserts by combination of the following procedures: PCR analysis, pattern of restriction enzyme digestion, and by insert sequencing. The resulting lentiviral vector also expresses GFP from a second promoter. The presence of GFP enables to follow siRNA expression in vitro via the appearance of glowing cells as well as in vivo in mouse model systems.

### EXAMPLE 2

### hPar1 Gene Silencing via SiRNA

i. The Effect of hPar1 siRNA on Tumor Growth and Invasive Properties

The effects of hPar1 silencing were measured both in a Matrigel invasion assay in vitro and in vivo in an experimental animal model.

The following highly metastatic human cancer cell lines were used MDA-231 breast cells, A375SM melanoma cells, or CL1 prostate carcinoma cells. These cells possess high levels of endogenous PAR1 (2, 23, 33).

Human MDA231, A375SM melanoma cells, or CL1 were transiently transfected with pL₂ or pL₄ or empty (mock) vectors by electroporation, and the cells were tested for PAR1 expression 48 hours later. Cells transfected with pL₂ or pL₄ contained 70%-80% less PAR1 mRNA than mock-transfected cells, as determined by semi quantitative RT-PCR (FIG. 2). In subsequent experiments, the effect of PAR1-targeted siRNA on CL1 invasiveness was tested using the “Matrigel invasion” assay. The ability of CL1 cells to invade through Matrigel-coated filters was inhibited in a statistically significant manner, 48 h after stable infection of hPar1 siRNA as compared to mock-infected cells. As demonstrated in FIG. 3 potent inhibition of Matrigel invasion is seen in the presence of hPar1 siRNA.

Next, the effect of siRNA-mediated PAR1 silencing on experimental metastasis in vivo was tested. For this purpose, tumor metastatic (CL1) cells were infected with pL₂ or the empty vector. After 48 h, when silencing of Par1 gene had reached its maximum, the cells were injected subcutaneously into nude mice (0.4×10⁶ cells per mouse). In this experimental metastasis model, the invasive properties of the tumor are critical for their extravasation, primarily during the first 3 h after the tumor cells enter into the circulation (34, 35). Therefore, the transient non-continuous silencing of PAR1 in either CL1 cells allows for the identification of parameters directly involved in blood-borne tumor dissemination such as the contribution of PAR1 to cell extravasation as opposed to its possible effects on subsequent survival of prostatic cancer s in the target organ, stromal support, and secondary metastasis. Thirty days after injection, the mice were killed and the primary tumors were evaluated by mass and histology.

FIG. 4 shows that hPar1 siRNA infected cells exhibit minimal tumor growth in vivo as compared to control vector infected cells that otherwise induce tumor growth. Specifically, FIG. 4(I) is a representative photograph of mice injected s.c. with CL1 cells that have been infected with either hPar1 siRNA vector (upper) or with control vector (lower). FIG. 4(II), tumor mass of CL1 cells infected with hPar1 siRNA vector relative to control vector is ˜15%.

FIG. 5 shows the results of GFP-hPar1siRNA infected CL1 cells, injected subcutaneously to SCID mice. The size of tumors formed was compared with CL1 cells injected alone (Vector).

ii. The Effect of hPar1 siRNA on Tumor Angiogenic Blood Vessel Formation:

Formation of new blood vessels is a critical determinant of tumor progression. hPar1 induces tumor angiogenesis partly via the over-expression of VEGF (24). Silencing of hPar1 may ultimately affect blood vessel angiogenic process.

The effect of hPar1 siRNA on tumor angiogenesis following the infection of bovine aortic endothelial cells (BAEC) as compared with mock-transfected cells was tested. These cells were assayed by the tube network formation assay and also by the Matrigel plug assay as previously described (24). Briefly, BAEC before and after hPar1 siRNA infection were embedded in a three dimensional collagen (type I) mesh and the extent of tube network formation was evaluated. Matrigel plug assay was performed with A375SM cells before and after hPar1 siRNA infection. In this assay it was possible to evaluate whether Par1 silencing inhibits the recruitment f blood vessels in vivo. The manipulated cells were mixed with Matrigel and injected s. c. into BALB/c mice. Upon injection, the liquid Matrigel rapidly formed a solid gel plug that serves not only as an inert vehicle for PAR1 producing cells but also mimicking the natural interactions that exist between tumor cells and the surrounding extra cellular matrix (ECM). Ten days post injection the Matrigel plugs were exposed, sliced paraffin-embedded and stained for the presence of blood vessels (vWF antibodies or Mallory's staining for collagen). Mature vs. sprouting angiogenic vessels were determined by staining for pericytes present on the surface of mature vessels only. VEGF ELISA were carried out for quantitative VEGF as previously described (24).

PAR1 induces VEGF and thereby promotes angiogenesis. Therefore, silencing of the gene is effective also for treatment of angiogenesis.

iii. The Effect of hPar1 siRNA on Tumor Metastasis in vivo:

In order to obtain metastasis in vivo, ortothopic injections to the prostate gland lobes were carried out. This enables the assessment of hPar1 siRNA impact on tumor growth & metastasis in an experimental model of prostate cancer.

An orthotopic mouse prostate treatment model: Athymic male nude mice (mu-mu; 6-8 weeks of age) were used. Orthotopic implantation (by an expert physician Dr. Ofer Gofrit, Urology Dept. Hadassah—Hebrew University Hospital) was carried out as described (10). Briefly, after total body anesthesia with ketamine (50 mg/kg) and xylazine (1-0 mg/kg), a low midline incision was made in the lower abdomen. A suspension of CL1 cells (5×10⁴) in 30 μl of PBS was injected into the lateral lobe of the prostate, and the wound was closed with surgical metal clips. This cell concentration was necessary to achieve consistent local tumor growth within 7 days of implantation.

One group of mice was injected with cells expressing the empty control vector and the other with hPar1 siRNA expressing cells. In parallel, another experiment was performed whereby the tumor was allowed to develop for 2 weeks prior to administration of hPar1 siRNA vector directly into the prostate gland lobes. For this purpose, mice were divided into several groups of treatment with 6 mice per treatment group. At day 7 and 14 post implantation, a low midline incision was performed, and the tumors were injected with plasmid constructs expressing hPar1 siRNA, or LL/EV (lentiviral LL/empty vector/EV) (75-100 μg). Fluorescence whole-body optical imaging (FIG. 6) revealed a progressive increase in the primary tumor and multiple metastatic growth in CL1/LL/EV cells but very little in the hPar1 siRNA treatment (representative for both groups). Specifically, fluorescent primary tumors were visible as early as 7 days after implantation (data not shown). The non invasive quantitative measurements of the externally visible fluorescent area and total fluorescent intensity may enable to construct in vivo tumor growth curve showing a linear tumor growth rate in the non treated aggressive tumor cell implanted (e.g., CL/LL/EV). In contrast, a marked lowering of the tumor fluorescence intensity and area was observed in the animals treated with the hPar1 siRNA expression vectors. Metastases were observed mainly in lymph nodes, liver, and the entire peritoneal region by 8 weeks following implantation.

1. A synthetic, small interfering nucleic acid (siNA) molecule, comprising a double stranded portion having a sequence capable of down regulating expression of a human protease-activated receptor 1 (PAR 1) gene via RNA interference.

2. A molecule according to claim 1 wherein said siNA is a small interfering ribonucleic acid (siRNA).

3. The molecule of claim 1 wherein each strand of said double stranded portion is about 18 to about 28 nucleotides in length.

4. The molecule of claim 3 wherein each strand of said double-stranded portion is about 19 nucleotides in length.

5. The molecule of claim 1, wherein one strand of said double stranded portion comprises an antisense region comprising a nucleotide sequence that is complementary to a nucleotide sequence of a hPAR 1 gene or a portion thereof, and wherein a second strand of said double stranded portion comprises a sense region comprising a nucleotide sequence that is substantially similar to a nucleotide sequence of said hPAR 1 gene or a portion thereof.

6. The molecule of claim 5 wherein said molecule is assembled from two separate nucleotide strands, wherein said separate strands form a double stranded molecule upon hybridization.

7. The molecule of claim 5, wherein the one strand is connected to the second strand of said double stranded portion via a linker.

8. The molecule of claim 7, wherein said linker is a polynucleotide linker.

9. The molecule of claim 5 wherein said molecule is assembled from a single nucleotide strand comprising said sense region, said antisense region and a polynucleotide linker; and wherein said sense region and said antisense region of said single nucleotide strand hybridize to form a double-stranded small hairpin nucleic acid structure.

10. A synthetic molecule according to claim 5 wherein the sense sequence comprises any of the sequences denoted as SEQ ID Nos. 1-4.

11. A synthetic molecule according to claim 9 wherein the single nucleotide strand comprises any of the sequences denoted as SEQ ID Nos. 5-12.

12. A pharmaceutical composition comprising the molecule of claim 1 in a pharmaceutically acceptable carrier or diluent.

13. A vector capable of transcribing one or more RNA molecules that self-hybridize or hybridize to each other to form a short hairpin RNA or short interfering RNA that inhibits expression of hPar1 in a cell.

14. The vector of claim 13, wherein the vector provides a template for synthesis of an RNA molecule that self-hybridizes to form a shRNA.

15. The vector of claim 13, wherein the vector provides a template for synthesis of complementary RNA molecules that hybridize with each other to form a siRNA.

16. The vector of claim 13 wherein the shRNA or siRNA comprise a double stranded region about 18 to about 28 nucleotides in length.

17. The vector of claim 16 wherein the double stranded region is approximately 19 nucleotides in length.

18. A vector in accordance with claim 14 comprising the nucleotide sequences denoted as SEQ ID Nos. 5-12.

19. A vector in accordance with claim 15 comprising the nucleotide sequences denoted as SEQ ID Nos. 1-4.

20. A vector according to claim 12, wherein the vector is pLentilox 3.7.

21. A pharmaceutical composition comprising the vector of claim 13 in a pharmaceutically acceptable carrier or diluent.

22. A host cell transfected with the vector of claim 13.
